(19) United States
(12) Patent Application Publication
(10) Pub. No.: US 2007/0276240 A1
(43) Pub. Date: Nov. 29, 2007

(54) System and method for imaging a target medium using acoustic and electromagnetic energies

(76) Inventors: S. Jeffrey Rosner, Palo Alto, CA (US); Claude Cohen-Bacrie, Ventabren (FR); Jacques Souquet, Puyricard (FR)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/416,525

(22) Filed: May 2, 2006

(51) Int. Cl. A61B 8/00

(52) U.S. Cl. 600/437

(57) ABSTRACT

A system and method for imaging a target medium uses both acoustic energy, e.g., ultrasound energy, and electromagnetic energy, e.g., microwave energy. The acoustic and electromagnetic energies are transmitted into the target medium using a transducer array of acoustic and electromagnetic transducers, which may also be used to receive echoes of the transmitted acoustic and electromagnetic energies from the target medium.

### BACKGROUND OF THE INVENTION

There are a number of imaging technologies available to visualize internal structures of a target medium using either electromagnetic or acoustic waves. These conventional imaging technologies are widely used for applications in many different fields, such as security, non-destructive testing, geological study and medicine. A particular application of interest for these imaging technologies is detection of breast cancer, which affects a significant percentage of the world population.

The most widely used imaging technology to detect breast cancer is mammography, which is the process of imaging a breast using low dose X-rays to detect tumors and cysts; x-ray imaging is sensitive to variations in density of the tissue. Mammography involves compressing a breast between two plastic plates to even out the tissue for better imaging and to hold the breast still for motion blur prevention. The actual detection of tumors and cysts requires the trained eyes of a radiologist to interpret the resulting X-ray images, also known as mammograms.

Although mammography is a powerful tool in early detection of breast cancer, there are several concerns with mammography. One of these concerns is that mammography produces a significant number of false negatives, which allows the breast cancer to progress. Another concern is that mammography produces a high rate of false positives, which can lead to unnecessary, invasive and costly biopsies.

Due to the high rate of false positives, mammography is commonly used as the first screening procedure for detection of breast cancer. For suspicious mammograms, one or more additional procedures are usually performed using different imaging technologies, such as acoustic imaging, magnetic resonance imaging (MRI), computed tomography (CT), and positron emission tomography (PET). The most common follow-up procedure for suspicious mammograms is ultrasound imaging, which is the most common technique among acoustic imaging techniques. Ultrasound imaging involves the use of high frequency acoustic pressure waves, which are usually transmitted into a subject using a handheld probe. When the high frequency acoustic waves encounter a boundary of different materials, such as fluid, soft tissue and bone, some of the acoustic waves are reflected back into the probe. The intensity and travel time of the reflected acoustic waves are used to produce an electronic image on a display. While ultrasound imaging works well to detect differences in mechanical properties, such as density and modulus, ultrasound imaging does not work well to detect differences in electrical properties, such as polarizability and conductivity.

Microwave imaging has also been proposed as a follow-up procedure to further assess suspicious mammograms, but is not currently in practice. Microwave imaging involves the use of non-ionizing electromagnetic waves in the frequency range from 10s of megahertz to 100s of gigahertz, i.e., microwaves, which are transmitted into a subject using an array of transceiving antennas or an array of receiving antennas and transmitting antennas. When the transmitted microwaves encounter a boundary of different materials, some of the transmitted microwaves are scattered back to the antenna array. The scattered microwaves are used to produce an electronic image on a display, which generally represents a two-dimensional (2D) slice of a three-dimensional (3D) image. In addition to medical applications, microwave imaging has been used in many other applications, such as security inspection for contraband, ground-penetrating radar for geology and mine detection, and, of course, commercial radar. In contrast to ultrasound imaging, microwave imaging works well to detect differences in electrical properties of materials, but does not work as well to detect differences in structural properties of materials.

In view of the above-described limitations in ultrasound and microwave imaging, there is a need for a system and method for imaging a target medium that can effectively detect differences in structural properties, as well as differences in electrical properties.

### SUMMARY OF THE INVENTION

A system and method for imaging a target medium uses both acoustic energy, e.g., ultrasound energy, and electromagnetic energy, e.g., microwave energy. The acoustic and electromagnetic energies are transmitted into the target medium using a transducer array of acoustic and electromagnetic transducers, which may also be used to receive reflections or attenuated versions of the acoustic energy and scattering of the electromagnetic energy from the target medium. The combined use of acoustic and electromagnetic energies provides enhanced detection of different internal materials of the target medium, which improves the information content of the resulting images of the target medium.

An imaging system in accordance with an embodiment of the invention comprises a transducer array, an acoustic transceiving unit, an electromagnetic transceiving unit and a processing unit. The transducer array comprises an acoustic transducer operable to transmit acoustic energy into the target medium in response to a first stimulus, an acoustic transducer operable to receive from the target medium an echo of the acoustic energy and to generate a first electrical signal in response thereto, an electromagnetic transducer operable to transmit electromagnetic energy into the target medium in response to a second stimulus, and an electromagnetic transducer operable to receive from the target medium an echo of the electromagnetic energy and to generate a second electrical signal in response thereto. The acoustic transceiving unit is connected to the transducer array to provide the first stimulus thereto and to receive the first electric signal therefrom. The electromagnetic transceiving unit is connected to the transducer array to provide the second stimulus thereto and to receive the second electrical signal therefrom. The processing unit is connected to the acoustic and electromagnetic transceiving units and operable to produce an image of the target medium in response at least in part to the first and second electrical signals.

A method for imaging a target medium in accordance with an embodiment of the invention comprises transmitting acoustic energy and electromagnetic energy into the target medium, receiving echoes of the acoustic energy and echoes of the electromagnetic energy from the target medium, generating respective electrical signals in response to the echoes of the acoustic energy and the echoes of the electromagnetic energy received from the target medium, and processing the electrical signals to produce an image of the target medium.

Other aspects and advantages of the present invention will become apparent from the following detailed description, taken in conjunction with the accompanying drawings, illustrated by way of example of the principles of the invention.

### BRIEF DESCRIPTION OF THE DRAWINGS

FIG. 1 is a block diagram of an imaging system in accordance with an embodiment of the invention.

FIGS. 2-4 are plan views illustrating different arrangements of ultrasound transducer elements and microwave antennas in a scan head of the imaging system of FIG. 1 in accordance with an embodiment of the invention.

FIG. 5 is a plan view illustrating a one-dimensional (1D) array of ultrasound transducer elements and microwave antennas in the scan head of the imaging system of FIG. 1 in accordance with an alternative embodiment of the invention.

FIG. 6A is a cross-sectional view of a stacked arrangement of ultrasound transducer elements and microwave antennas in the scan head of the imaging system of FIG. 1 in accordance with an embodiment of the invention.

FIG. 6B is a cross-sectional view of another stacked arrangement of ultrasound transducer elements and microwave antennas in the scan head of the imaging system of FIG. 1 in accordance with an embodiment of the invention.

FIG. 7A is a block diagram illustrating components of ultrasound and microwave transceiving units included in the imaging system of FIG. 1 in accordance with an embodiment of the invention.

FIG. 7B is a block diagram illustrating components of ultrasound and microwave transceiving units included in the imaging system of FIG. 1 in accordance with an alternative embodiment of the invention.

FIG. 8 is a block diagram of an imaging system in accordance with another embodiment of the invention.

FIGS. 9-12 are plan views illustrating different arrangements of ultrasound transducer elements and microwave antennas in a stationary scan head of the imaging system of FIG. 8 in accordance with an embodiment of the invention.

FIG. 13 is a process flow diagram of a method for imaging a target medium in accordance with an embodiment of the invention.

### DETAILED DESCRIPTION

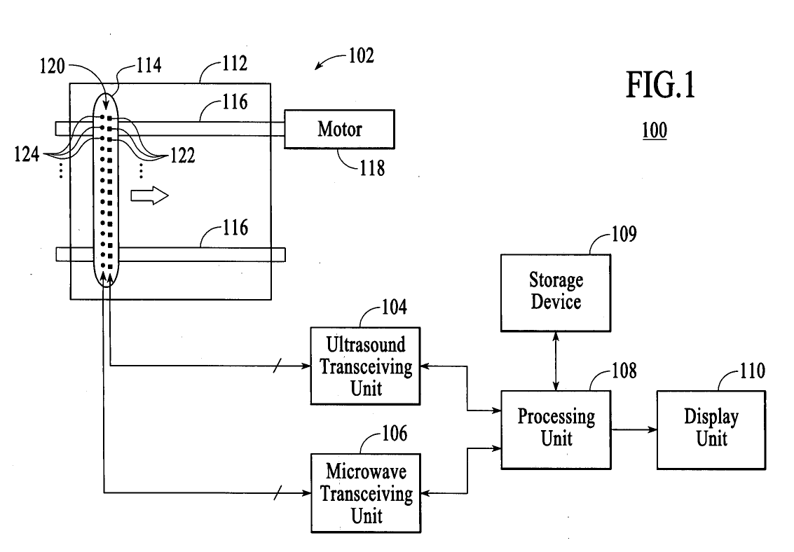

With reference to FIG. 1, an imaging system 100 in accordance with an embodiment of the invention is described. The imaging system 100 uses both ultrasound and microwave imaging technologies, which enhances the performance of the imaging system. Ultrasound imaging technology is sensitive to structural properties, such as density and modulus, while microwave imaging technology is sensitive to electrical properties, such as polarizability and conductivity. Thus, the imaging system 100 is sensitive to both the structural properties and the electrical properties of internal structures of a target medium to better differentiate between the different internal structures of the target medium.

The imaging system 100 is described herein as being used for breast cancer detection. However, the imaging system 100 may be used for other medical imaging applications, as well as non-medical imaging applications, such as non-destructive testing and security inspection. Furthermore, although the imaging system 100 is described herein as using ultrasound and microwave energies, i.e., ultrasound waves and microwaves, the imaging system may use other acoustic and electromagnetic energies.

As shown in FIG. 1, the imaging system 100 includes a dual-mode scanning unit 102, an ultrasound transceiving unit 104, a microwave transceiving unit 106, a processing unit 108, a storage device 109 and a display unit 110. The dual-mode scanning unit 102 is used to scan a target medium of interest, which may be a breast of a person for breast cancer detection, using both ultrasound and microwave energies. The entire target medium is scanned by sequentially scanning two-dimensional (2D) slices of the target medium using ultrasound and microwave energies to image the internal structures of the target medium. The resulting image can then be used to detect cysts and tumors in a breast for cancer detection. As described in more detail below, the dual-mode scanning unit 102 includes ultrasound transducer elements 122 that transmit and receive ultrasound energy and microwave transducer elements in the form of microwave antennas 124 that transmit and receive microwave energy. The ultrasound transceiving unit 104 is configured to provide driving signals to the ultrasound transducer elements 122 of the scanning unit 102. The driving signals control the transmission of ultrasound energy from the scanning unit into a target medium. The ultrasound transceiving unit 104 is also configured to receive electrical signals from the ultrasound transducer elements 122 of the scanning unit. The electrical signals are generated by the ultrasound transducer elements in response to echoes of the transmitted ultrasound energy received from the target medium. The ultrasound transceiving unit 104 is further configured to produce summed electrical signals in response to the received electrical signals from the ultrasound transducer elements 122. Each summed electrical signal is derived from the electrical signals that represent an ultrasound echo from a focused point in the target medium, which is further described below.

Similarly, the microwave transceiving unit 106 is configured to provide driving signals to the microwave antennas 124 of the scanning unit 102. The driving signals control the transmission of microwave energy from the scanning unit 102 into the target medium. The microwave transceiving unit 106 is also configured to receive electrical signals from the microwave antennas 124 of the scanning unit 102. These electrical signals are generated by the microwave antennas in response to echoes of the transmitted microwave energy received from the target medium. The microwave transceiving unit 106 is further configured to produce summed electrical signals in response to the received electrical signals from the microwave antennas 124. Each summed electrical signal is derived from the electrical signals that represent a microwave echo from a focused point in the target medium, which is further described below.

The processing unit 108 processes the summed electrical signals from the ultrasound and microwave transceiving units 104 and 106 to generate one or more images of the target medium. The images are typically three-dimensional (3D) images. As the target medium is scanned by the scanning unit 102, the processing unit 108 stores the acquired information for each 2D slice of the target medium in the storage device 109. The storage device 109 may be any type of a data storage device, such as a computer hard drive. The images of the target medium generated by the processing unit 108 are electronically displayed on the display unit 110. The acoustic and microwave 3D images may be displayed to correspond exactly to provide an overlay of the information from the two modalities in each slice view. The images may be displayed together using several color channels, e.g. red for microwave boundaries, blue for acoustic boundaries, and purple for regions of both microwave and acoustic reflectance.

As illustrated in FIG. 1, the dual-mode scanning unit 102 includes a scanning plate 112, a scan head 114, tracks 116 and a motor 118. The scanning plate 112 is used to interface with the target medium. Typically, the scanning l s plate 112 is made of a material that minimizes the reflection of ultrasound and microwave energies at the interface of the scanning plate and the target medium. The scan head 114 is positioned such that the scanning plate 112 is between the scan head and the target medium. The scan head 114 is mounted on the tracks 116 and can be linearly displaced along the tracks by the motor 118. The scan head 114 includes an array 120 of ultrasound transducer elements 122 and microwave antennas 124. In the illustrated embodiment, the array 120 includes one column of ultrasound transducers 122 and one column of microwave antennas 124. Each ultrasound transducer element 122 can transmit and receive ultrasound energy. Similarly, each microwave antenna 124 can transmit and receive microwave energy. Thus, in this embodiment, the ultrasound transducer elements 122 and the microwave antennas 124 of the array 120 are used to both transmit and receive the respective energy. However, in other embodiments, the ultrasound transducer elements 122 and the microwave antennas 124 of the array 120 are used to either exclusively transmit or exclusively receive the respective energy. In these embodiments, the array 120 includes an additional column of ultrasound transducer elements (not shown). One of the two columns of ultrasound transducer elements is used to transmit ultrasound energy, while the other column of ultrasound transducer elements is used to receive echoes of the transmitted ultrasound energy. Similarly, in these embodiments, the array 120 includes an additional column of microwave antennas (not shown). One of the two columns of microwave antennas is used to transmit microwave energy, while the other column of microwave antennas is used to receive echoes of the transmitted microwave energy.

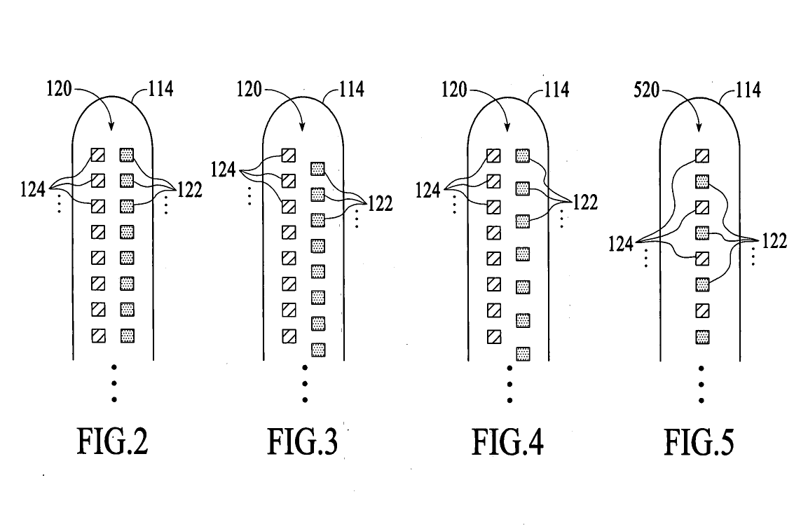

In the array 120, the ultrasound transducer elements 122 and the microwave antennas 124 can be positioned in different arrangements. In one arrangement, as illustrated in FIG. 2, each ultrasound transducer element 122 is positioned next to one of the microwave antennas 124. In another arrangement, as illustrated in FIG. 3, the ultrasound transducer elements 122 and the microwave antenna 124 are positioned in a staggered configuration. In another arrangement, as illustrated in FIG. 4, the ultrasound transducer elements 122 and the microwave antenna 124 are positioned such that the pitch of acoustic transducer elements differs from the pitch of the microwave antennas 124. In other arrangements, the array 120 includes additional columns of ultrasound transducer elements 122 and additional columns of microwave antennas 124, as described above.

In an alternative embodiment, illustrated in FIG. 5, the scan head 114 includes a one-dimensional (1D) array 520 of ultrasound transducer elements 122 and microwave antennas 124. Thus, the array 520 includes only a single column of ultrasound transducer elements 122 and microwave antennas 124 in which the ultrasound transducer elements 122 and the microwave antennas 124 are interleaved. Alternatively, more than one ultrasound transducer element 122 may be positioned between two adjacent microwave antennas 124, or more than one microwave antenna may be positioned between two adjacent microwave antennas.

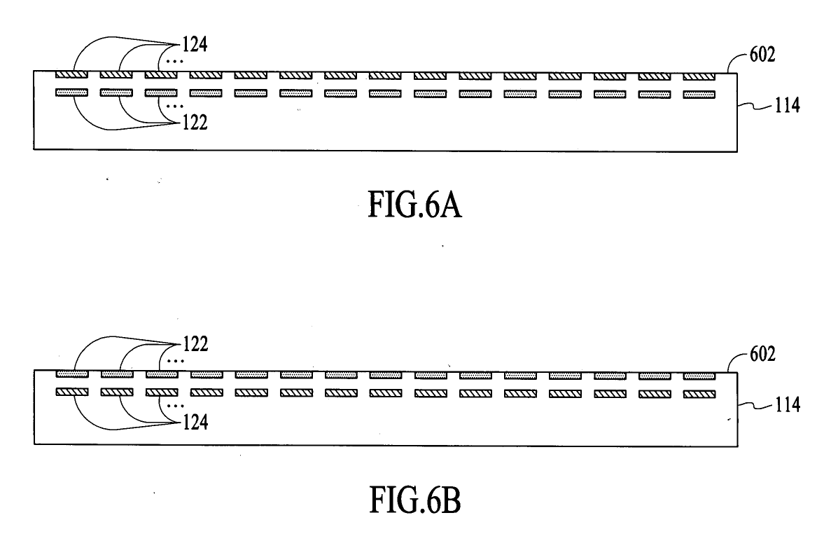

In another alternative embodiment, the ultrasound transducer elements 122 and the microwave antennas 124 are stacked, as illustrated in FIGS. 6A and 6B. FIGS. 6A and 6B are cross-sectional views of the scan head 114. In one arrangement, as shown in FIG. 6A, the microwave antennas 124 are located at or near a surface 602 of the scan head 114 that interfaces the target medium, and each ultrasound transducer element 122 is positioned below one of the microwave antennas. Thus, the ultrasound transducer elements 124 are closer to the target medium than the microwave antennas 122. In another arrangement, as shown in FIG. 6B, the ultrasound transducer elements 122 are located at or near the surface 602 of the scan head 114, and each microwave antenna 124 is positioned below a respective one of the ultrasound transducer elements. Thus, the microwave antennas 122 are closer to the target medium than the ultrasound transducer elements 124. In either of these arrangements, the scan head 114 is made of a material that has mechanical properties that match the ultrasound transducer elements 122 to the target medium and dielectric properties that match the microwave antennas 124 to reduce the reflection of energy at the interface with the target medium. Although the ultrasound transducer elements 122 and the microwave antennas 124 are shown spatially separated in FIGS. 6A and 6B, the ultrasound transducer elements may physically contact the respective microwave antennas.

Turning back to FIG. 1, the ultrasound transceiving unit 104 is electrically connected to the ultrasound transducer elements 122 of the scanning unit 102 to provide driving signals to the ultrasound transducer elements and to receive electrical signals from the ultrasound transducer elements. The driving signals control the transmission of ultrasound energy from the scanning unit 102 into a target medium. The electrical signals received from the ultrasound transducer elements 122 are generated by the ultrasound transducer elements in response to echoes of the transmitted ultrasound energy received from the target medium. Similarly, the microwave transceiving unit 106 is electrically connected to the microwave antennas 124 of the scanning unit 102 to provide driving signals to the microwave antennas and to receive electrical signals from the microwave antennas. The driving signals control the transmission of microwave energy from the scanning unit into the target medium. The electrical signals received from the microwave antennas are generated by the microwave antennas in response to echoes of the transmitted microwave energy received from the target medium.

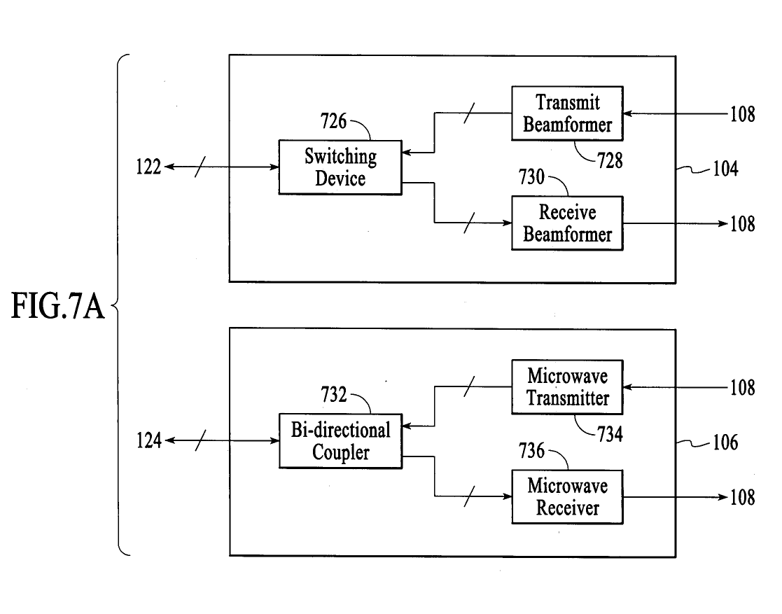

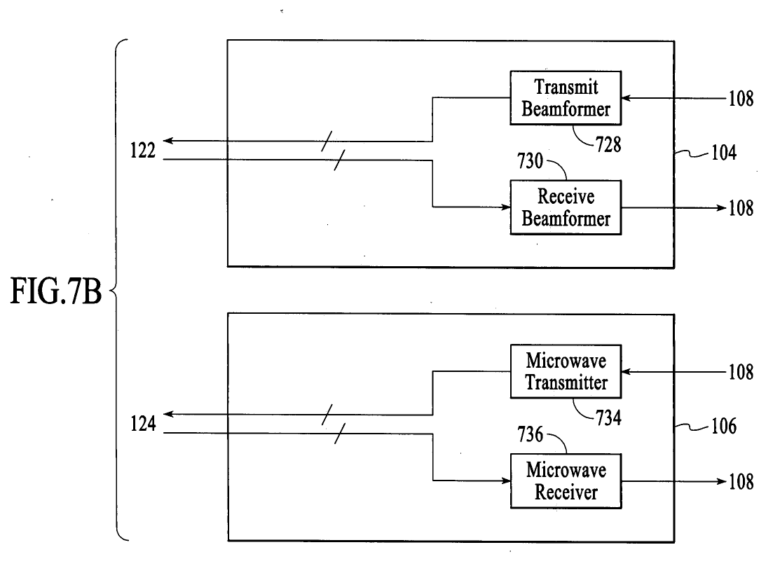

Turning now to FIG. 7A, the components of the ultrasound transceiving unit 104 and the microwave transceiving unit 106 in accordance with an embodiment of the invention are shown. In this embodiment, each of the ultrasound transducer elements 122 and the microwave antennas 124 of the scanning unit 102 is used to both transmit and receive the respective energy. Thus, each of the ultrasound transducer elements 122 is a transceiving ultrasound transducer element and each of the microwave antennas 124 is a transceiving microwave antenna. As illustrated in FIG. 7A, the ultrasound transceiving unit 104 includes a switching device 726, a transmit beamformer 728 and a receive beamformer 730. The switching device 726 connects the ultrasound transducer elements 122 to either the transmit beamformer 728 or the receive beamformer 730 to transmit and receive electrical signals to and from the ultrasound transducer elements.

The transmit beamformer 728 drives the individual ultrasound transducer elements 122 of the scanning unit 102 using stimuli in the form of electrical signals. The electrical signals cause the ultrasound transducer elements 122 to generate ultrasound energy, which is transmitted into the target medium. In an embodiment, the transmit beamformer 728 drives the individual ultrasound transducer elements 122 in a manner that causes the ultrasound transducer elements to generate ultrasound energy, which is focused at points within the target medium along a linear path to form a narrow ultrasound beam on a scanning 15 plane. The scanning plane is a plane that extends through the ultrasound transducer elements 122 and is orthogonal to the plane on which the scan head 114 is linearly displaced. The ultrasound beam is produced by the constructive interference of the ultrasound energy from the different transducer elements 122. The focusing of the ultrasound energy from the ultrasound transducer elements 122 is achieved by selectively activating the ultrasound transducer elements in a predefined timing sequence using activation electrical signals transmitted from the transmit beamformer 728 to the ultrasound transducer elements via the switching device 726. The activation electrical signals drive the individual ultrasound transducer elements 122 to generate ultrasound energy, which is transmitted into the target medium. The ultrasound beam can also be steered to different direction on the scanning plane so that the ultrasound energy can be focused at different points within the target medium throughout the scanning plane to acquire imaging information on a 2D slice of the target medium. The steering of the ultrasound beam can be achieved by selectively activating the ultrasound transducer elements 122 using different timing sequences so that the ultrasound energy is focused at points within the target medium along various linear directions.

Echoes of the transmitted ultrasound energy are received by the ultrasound transducer elements 122 from the target medium. In response to the echoes, the ultrasound transducer elements 122 generate respective “ultrasound” electrical signals that represent the received-echoes. The ultrasound electrical signals are transmitted to the receive beamformer 730 via the switching device 726. Since an ultrasound echo of the ultrasound beam from a particular focused point within the target medium arrives at the individual ultrasound transducer elements 122 at different times, the receive beamformer 730 provides delays so that the ultrasound electrical signals corresponding to the ultrasound echo from that particular point can be combined to produce a summed ultrasound electrical signal. Using different delays, summed ultrasound electrical signals for points throughout the scanning plane can be produced. The summed ultrasound electrical signals are transmitted to the processing unit 108 where the signals are processed to form a 2D slice image of the target medium.

The above process of transmitting ultrasound energy and receiving ultrasound echoes is repeated as the ultrasound beam is steered on a particular scanning plane to acquire imaging information for one 2D slice image of the target medium. The entire process is then repeated as the scan head 114 is displaced step-wise along the tracks 116 by the motor 118 to scan additional 2D slices of the target medium. This process of transmitting and receiving ultrasound energy is commonly known as a phased array technique. However, in other embodiments, different techniques may be employed to image the target medium using ultrasound energy.

The microwave transceiving unit 106 includes a bi-directional coupler 732, a microwave transmitter 734 and a microwave receiver 736. The bi-directional coupler 732 connects the microwave antennas 124 to the microwave transmitter 734 and the microwave receiver 736 to transmit and receive electrical signals to and from the microwave antennas. In other embodiments, the bi-directional coupler 732 may alternatively be a circulator.

The microwave transmitter 734 drives the individual microwave antennas 124 using stimuli in the form of electrical signals so that the microwave antennas generate microwave energy, which is transmitted into the target medium. The microwave transmitter 734 generates the electrical signals with a frequency in the microwave range. The electrical signals are transmitted to the microwave antennas 124 via the bi-directional coupler 732 to drive the individual microwave antennas. In response to these electrical signals, the microwave antennas 124 generate and transmit microwave energy. Similar to the transmit beamformer 728 of the ultrasound transceiving unit 104, in an embodiment, the microwave transmitter 734 transmits the electrical signals in different timing sequences to focus and steer the microwave energy generated by the individual microwave antennas 124.

Echoes of the transmitted microwave energy are received by the microwave antennas 124. In response to the echoes, the microwave antennas 124 generate “microwave” electrical signals that represent the received microwave echoes. The microwave electrical signals are transmitted to the microwave receiver 736 via the bi-directional coupler 732. Similar to the receive beamformer 730 of the ultrasound transceiving unit 104, in an embodiment, the microwave transmitter 734 provides delays so that the microwave electrical signals corresponding to each focused point in the target medium can be combined to produce a summed microwave electrical signal.

In other embodiments, the transmission and reception of microwave energy may alternatively be performed in accordance with a beam steering technique in which microwave energy is transmitted by all the microwave -antennas 124 to form a directional beam but only one of the microwave antennas is used to receive the microwave echoes. Alternatively, some of the microwave -antennas 124 may be sequentially activated to transmit microwave energy and some of the non-transmitting microwave antennas may be used to receive the microwave echoes. Similar to the process of transmitting ultrasound energy and receiving ultrasound echoes, the process of transmitting microwave energy and receiving microwave echoes is repeated as the microwave beam is steered on a particular scanning plane, and then the entire process is repeated as the scan head 114 is displaced step-wise along the tracks 116 by the motor 118 to scan the target medium.

At each position of the scan head 114 along the tracks 116, the process of transmitting ultrasound energy and receiving ultrasound echoes and the process of transmitting microwave energy and receiving microwave echoes may be performed simultaneously. Alternatively, the two processes may be performed sequentially. If performed sequentially, either of the two processes may be performed first.

Turning now to FIG. 7B, the components of the ultrasound transceiving unit 104 and the microwave transceiving unit 106 in accordance with an alternative embodiment of the invention are shown. In this alternative embodiment, each of the ultrasound transducer elements 122 and the microwave antennas 124 of the scanning unit 102 is used to either transmit or receive the respective energy exclusively. Thus, each of the ultrasound transducer elements 122 is either a transmitting ultrasound transducer element or a receiving ultrasound transducer element, and each of the microwave antennas 124 is either a transmitting microwave antenna or a receiving microwave antenna. Consequently, the switching device 726 of the ultrasound transceiving unit 104 and the bidirectional coupler 732 of the microwave transceiving unit 106 are not needed in this embodiment. Rather, the transmit beamformer 728 of the ultrasound transceiving unit 104 is connected directly to the transmitting ultrasound transducer elements 122 and the receive beamformer 730 is connected directly to the receiving ultrasound transducer elements 122. Similarly, the microwave transmitter 734 of the microwave transceiving unit 106 is connected directly to the transmitting microwave antennas 124 and the microwave receiver 736 is connected directly to the receiving microwave antennas 124.

Turning back to FIG. 1, the processing unit 108 of the imaging system 100 processes the summed ultrasound electrical signals from the receive beamformer 730 of the ultrasound transceiving unit 104 and the summed microwave electrical signals from the microwave receiver 736 of the microwave transceiving unit 106 to produce one or more 3D images of the target medium. The images are displayed on the display unit 110 or stored in the storage device 109 for display at a later time. At each position of the scan head 114 along the tracks 116, as ultrasound and microwave beams are transmitted into the target medium and scanned in a new scanning plane, the processing unit 108 receives summed ultrasound and microwave electrical signals. These electrical signals collectively represent a 2D image slice of the target medium along that scanning plane. As the scan head 114 is stepped along the tracks 116 by the motor 118, additional summed ultrasound and microwave electrical signals are received and processed by the processing unit 108 to produce respective 2D image slices. The 2D image slices are then combined to produce one or more 3D images of the target medium. The 3D images can be displayed on the display unit 110. For applications in breast cancer detection, the resulting 3D images can be examined to identify cysts and tumors.

The processing unit 108 also provides control signals to the motor 118, the ultrasound transceiving unit 104 and the microwave transceiving unit 106. The control signals to the motor 118 control the step-wise displacement of the scan head 114 along the tracks 116. The control signals to the ultrasound transceiving unit 104 control the transmitting of ultrasound energy and the processing of ultrasound electrical signals generated by the ultrasound transducer elements 122 in response to received ultrasound echoes. Similarly, the control signals to the microwave transceiving unit 106 controls the transmitting of microwave energy and the processing of microwave electrical signals generated by the ultrasound transducer elements 122 in response to received microwave echoes.

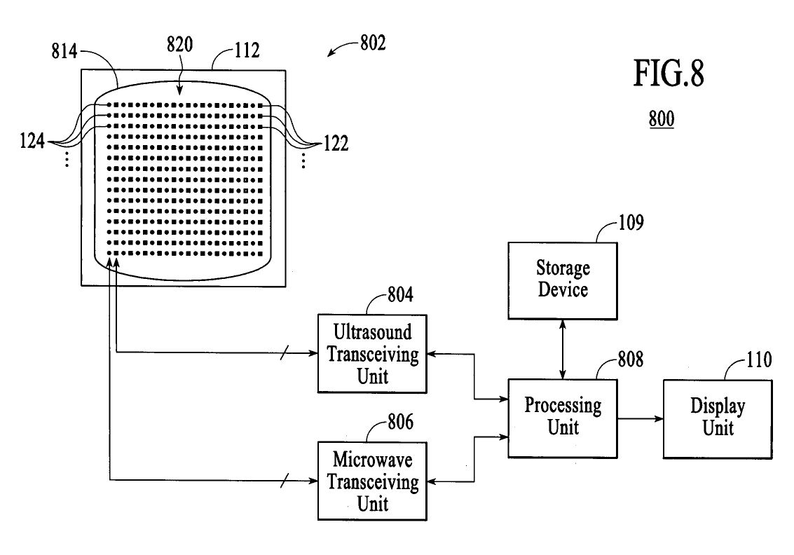

Turning now to FIG. 8, an imaging system 800 in accordance with another embodiment is shown. The same reference numerals used in FIG. 1 are used in FIG. 8 to identify similar elements. The imaging system 800 includes a dual-mode scanning unit 802 that uses a 2D array 820 of ultrasound transducer elements 122 and microwave antennas 124 on a stationary scan head 814 rather than the 1D array 120 or 520 mounted on the movable scan head 114 of the imaging system 100. The 2D array 820 of ultrasound transducer elements 122 and microwave antennas 124 allows a target medium to be imaged using ultrasound and microwave energy without having to move the array, as is the case for the imaging system 100.

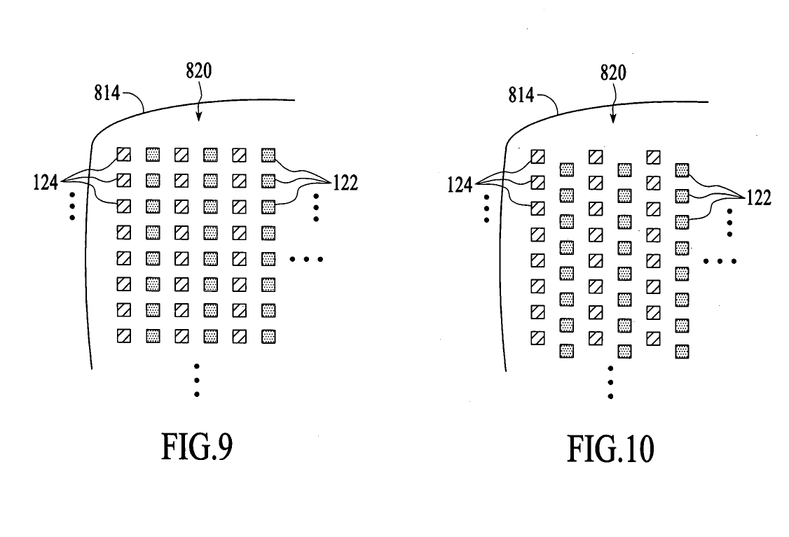

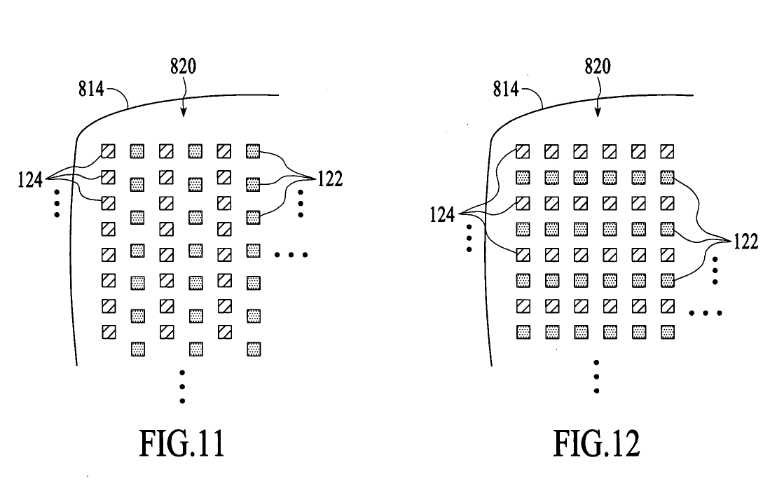

The dual-mode scanning unit 802 includes the scanning plate 112 and the 2D array 820 of ultrasound transducer elements 122 and microwave antennas 124 on the stationary scan head 814. The ultrasound transducer elements 122 and the microwave antennas 124 in the 2D array 820 may be positioned in different arrangements. In one arrangement, illustrated in FIG. 9, the 2D array 820 includes interleaved columns of ultrasound transducer elements 122 and columns of microwave antennas 124. In addition, the ultrasound transducer elements 122 and the microwave antennas 124 are positioned in the 2D array 820 such that each ultrasound transducer element is next to one of the microwave antennas. In another arrangement, illustrated in FIG. 10, the ultrasound transducer elements 122 and the microwave antennas 124 of adjacent columns in the 2D array 820 are positioned in a staggered configuration. In another arrangement, illustrated in FIG. 11, the ultrasound transducer elements 122 are positioned in the 2D array 820 such that the pitch of the ultrasound transducer elements of a given column differs from the pitch of the microwave antennas 124 of an adjacent column. In another arrangement, illustrated in FIG. 12, each of the columns of the 2D array 720 includes both ultrasound transducers 122 and microwave antennas 124. The ultrasound transducer elements 122 and the microwave antennas 124 of these “combination” columns may be arranged such that the ultrasound transducer elements 122 are interleaved with the microwave antennas 124. Alternatively, more than one ultrasound transducer element 122 may be positioned between two adjacent microwave antennas 124, or more than one microwave antenna may be positioned between two adjacent ultrasound transducer elements. In an alternative embodiment, the ultrasound transducer elements 122 and the microwave transducer elements 124 are stacked, as illustrated in FIGS. 6A and 6B.

The ultrasound transceiving unit 804 is connected to the ultrasound transducer elements 122 of the 2D array 820, while the microwave transceiving unit 806 is connected to the microwave antennas 124 of the 2D array 820. The ultrasound transceiving unit 804 is configured to control the transmission of ultrasound energy from the ultrasound transducer elements 122 of the 2D array 820 and to receive “ultrasound” electrical signals that are generated by the ultrasound transducer elements in response to received reflections of the transmitted ultrasound energy. Similarly, the microwave transceiving unit 806 is configured to control the transmission of microwave energy from the microwave antennas 124 of the 2D array 820 and to receive “microwave” electrical signals that are generated by the microwave antennas in response to the received microwave echoes.

In operation, the ultrasound and microwave transceiving units 804 and 806 selectively transmit activation electrical signals to the ultrasound transducer elements 122 and the microwave antennas 124 of the 2D array 820 to transmit ultrasound and microwave energies into the target medium. Echoes of the transmitted ultrasound and microwave energies from the target medium are then received by the ultrasound transducer elements 122 and the microwave antennas 124 of the 2D array 820. The transmitting and receiving of the respective energy may be sequentially performed by the same ultrasound transducer elements 122, i.e., the transceiving ultrasound transducer elements, or the same microwave antennas 124, i.e., the transceiving microwave antennas. Alternatively, the transmitting of the respective energy is performed by the transmitting ultrasound transducer elements 122 and the transmitting microwave antennas 124, while the receiving of the respective energy is performed by the receiving ultrasound transducer elements 122 and the receiving microwave antennas 124. The ultrasound and microwave transceiving units 804 and 806 can sequentially select a group of ultrasound transducer elements 122 and microwave antennas 124 of the 2D array 820 to scan the target medium. As an example, the ultrasound and microwave transceiving units 804 and 806 may sequentially select one or more columns of the ultrasound transducer elements 122 and the microwave antennas 124 in the 2D array 820 for transmission and reception of ultrasound and microwave energies to scan the target medium in a manner similar to the imaging system 100.

The processing unit 808 of the imaging system 800 processes signals generated by the ultrasound and microwave transceiving units 804 and 806 that represent the received ultrasound and microwave echoes to generate one or more images of the target medium. The images are typically three-dimensional (3D) images. These images of the target medium may be displayed on the display unit 110 or stored in the storage device 109 for subsequent display.

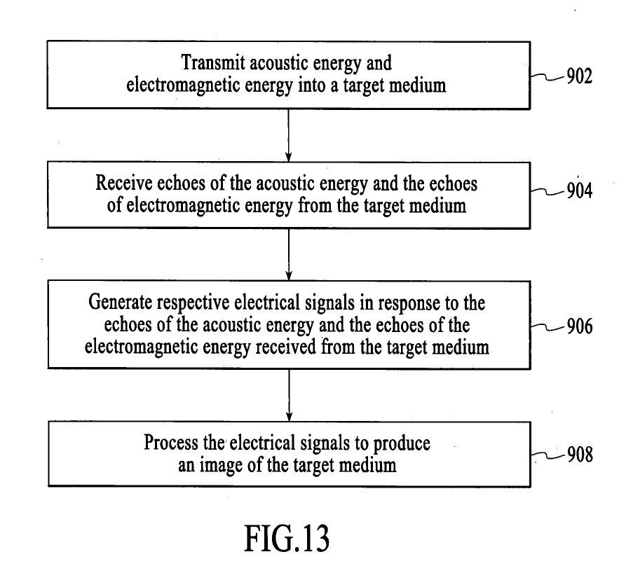

A method for imaging a target medium in accordance with an embodiment of the invention is described with reference to a process flow diagram of FIG. 13. At block 902, acoustic energy and electromagnetic energy are transmitted into the target medium. In an embodiment, the acoustic energy is ultrasound energy and the electromagnetic energy is microwave energy. Next, at block 904, echoes of the acoustic energy and echoes of the electromagnetic energy are received from the target medium. Next, at block 906, respective electrical signals are produced in response to the echoes of the acoustic energy and the echoes of the electromagnetic energy received from the target medium. Next, at block 908, the electrical signals are processed to produce an image of the target medium. In an embodiment, a transducer array of acoustic and electromagnetic transducers is used to transmit and receive the respective energy.

Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. A system for imaging a target medium, the system comprising:
a transducer array comprising an acoustic transducer operable to transmit acoustic energy into said target medium in response to a first stimulus, an acoustic transducer operable to receive from said target medium an echo of said acoustic energy and to generate a first electrical signal in response thereto, an electromagnetic transducer operable to transmit electromagnetic energy into said target medium in response to a second stimulus, and an electromagnetic transducer operable to receive from said target medium an echo of said electromagnetic energy and to generate a second electrical signal in response thereto;
an acoustic transceiving unit connected to said transducer array to provide said first stimulus thereto and to receive said first electric signal therefrom;
an electromagnetic transceiving unit connected to said transducer array to provide said second stimulus thereto and to receive said second electrical signal therefrom; and
a processing unit connected to said acoustic and electromagnetic transceiving units and operable to produce an image of said target medium in response at least in part to said first and second electrical signals.

2. The imaging system of claim 1, wherein said acoustic transducer comprises an ultrasound transducer and said electromagnetic transducer comprises a microwave transducer.

3. The imaging system of claim 1, wherein said acoustic transducer operable to transmit and said acoustic transducer operable to receive are the same acoustic transducer.

4. The imaging system of claim 1, wherein said electromagnetic transducer operable to transmit and said electromagnetic transducer operable to receive are the same electromagnetic transducer.

5. The imaging system of claim 1, wherein said acoustic transducer operable to transmit and said acoustic transducer operable to receive are different acoustic transducers.

6. The imaging system of claim 1, wherein said electromagnetic transducer operable to transmit and said electromagnetic transducer operable to receive are different electromagnetic transducers.

7. The imaging system of claim 1, wherein said transducer array comprises a column comprising acoustic transducers and electromagnetic transducers.

8. The imaging system of claim 1, wherein said transducer array comprises a column consisting of acoustic transducers and a column consisting of electromagnetic transducers.

9. The imaging system of claim 1, further comprising:
a scan head comprising said transducer array; and
a motor coupled to said scan head to step said scan head to scan said target medium.

10. The imaging system of claim 1, wherein one of said acoustic transducers and one of said electromagnetic transducers have a stacked arrangement.

11. The imaging system of claim 1, wherein said transducer array additionally comprises an additional acoustic transducer and an additional electromagnetic transducer.

12. A system for imaging a target medium, the system comprising:
a transducer array comprising an ultrasound transducer operable to transmit ultrasound waves into said target medium in response to a first stimulus, an ultrasound transducer operable to receive from said target medium an echo of said ultrasound energy and to generate a first electrical signal in response thereto, a microwave transducer operable to transmit microwave energy into said target medium in response to a second stimulus, and a microwave transducer operable to receive from said target medium an echo of said microwave energy and to generate a second electrical signal in response thereto;
an ultrasound transceiving unit connected to said transducer array to provide said first stimulus thereto and to receive said first electric signal therefrom;
a microwave transceiving unit connected to said transducer array to provide said second stimulus thereto and to receive said second electrical signal therefrom; and
a processing unit connected to said ultrasound and microwave transceiving units and operable to produce an image of said target medium in response at least in part to said first and second electrical signals.

13. The imaging system of claim 12, wherein said ultrasound transducer operable to transmit and said ultrasound transducer operable to receive are the same ultrasound transducer.

14. The imaging system of claim 12, wherein said microwave transducer operable to transmit and said microwave transducer operable to receive are the same microwave transducer.

15. The imaging system of claim 12, wherein said ultrasound transducer operable to transmit and said ultrasound transducer operable to receive are different ultrasound transducers.

16. The imaging system of claim 12, wherein said microwave transducer operable to transmit and said microwave transducer operable to receive are different microwave transducers.

17. The imaging system of claim 12, further comprising:
a scan head comprising said transducer array; and
a motor coupled to said scan head to step said scan head to scan said target medium.

18. The imaging system of claim 12, wherein said transducer array additionally comprises an additional ultrasound transducer and an additional microwave transducer.

19. A method for imaging a target medium, the method comprising:
transmitting acoustic energy and electromagnetic energy into said target medium;
receiving echoes of said acoustic energy and echoes of said electromagnetic energy from said target medium;
generating respective electrical signals in response to said echoes of said acoustic energy and said echoes of said electromagnetic energy received from said target medium; and
processing said electrical signals to produce an image of said target medium.

20. The method of claim 19, wherein said transmitting includes transmitting ultrasound energy and transmitting microwave energy into said target medium.

21. The method of claim 19, wherein said transmitting and said receiving are performed using a transducer array comprising an acoustic transducer and an electromagnetic transducer.

22. The method of claim 21, further comprising moving said array to scan said target medium.
